(19) United States
(12) Patent Application Publication
(10) Pub. No.: US 2006/0161232 A1
(43) Pub. Date: Jul. 20, 2006

(54) Phase-change particulate ice slurry coolant medical delivery tubing and insertion devices

(75) Inventors: Kenneth E. Kasza, Palos Park, IL (US); John Oras, Des Plaines, IL (US); HyunJin Son, Naperville, IL (US); Lance B. Becker, Chicago, IL (US); Terrry Vanden Hoek, Chicago, IL (US)

Correspondence Address: BRINKS HOFER GILSON & LIONE, P.O. BOX 10395, CHICAGO, IL, 60610, US

(73) Assignees: Kasza, Oras and Son to The University of Chicago; Kasza, Oras, Son, Becker and Vanden Hoek to The University of Chicago

(21) Appl. No.: 11/038,570

(22) Filed: Jan. 18, 2005

(51) Int. Cl. A61F 7/00; A61F 7/12

(52) U.S. Cl. 607/105; 607/113

(57) ABSTRACT

Various systems for delivering phase-change particulate ice slurries to targeted areas or organs of a patient are provided. Systems for delivering phase-change particulate ice slurries include a slurry reservoir and a conduit for delivering slurry from the reservoir to the patient. The conduit may include multiple components, including a section of medical tubing, an insertion tip for directing the out flow of slurry to the targeted area, and one or more transition fittings to adapt the tubing to the insertion tip. Interfaces between the various components that form the slurry flow path are configured so that there are no sudden reductions in the cross sectional area of the flow path. Any narrowing of the flow path occurs in a gradual tapered manner. The entire flow path remains substantially free of all obstacles that may tend to trap particles and lead to plugging of the flow path.

### FEDERALLY SPONSORED RESEARCH OR DEVELOPMENT

This invention was made with Government support under HIH HL 67630 from the National Institutes of Health. The Government may therefore have certain rights in this invention.

The United States Government has rights in this invention pursuant to contract Number w-31-109-ENG-38 between the United States Government and Argonne National Laboratory.

### BACKGROUND

The present invention relates generally to medical delivery systems. In particular, the invention relates to systems for delivering phase-change particulate slurries, such as ice slurry coolants to targeted areas or organs of the body.

Rapid inducement of protective hypothermia has been found to improve the survival rates of patients suffering from a variety of ailments. These include ischemia as a result of cardiac arrest, myocardial infarction, stroke, hemorrhage or traumatic injury and various medical procedures. More traditional methods for inducing protective hypothermia have included techniques such as ice water immersion; ice packs applied to a patient's head and torso; surface cooling of the head and neck; extracorporeal blood cooling; and cardio pulmonary bypass with a heat exchanger. More recently developed cooling techniques include endovascular heat exchange, and application of ice slurries to targeted areas or organs of the body.

Various methods for inducing hypothermia using phase-change particulate slurries are described in U.S. Pat. No. 6,597,811 to Becker et al., the entire disclosure of which is incorporated by reference in the present disclosure. According to the methods described by Becker et al., saline ice slurries, perfluorocarbon slurries or other types of slurries compatible with human tissue are used to directly cool various internal organs of the body. Ice slurries may be delivered to the body's internal heat exchangers, such as the lungs (endotracheal); G.I. (oral-gastric); carotid artery (peri-vascular); and peritoneal cavity (lavage). Ice slurries may also be delivered by direct intravenous insertion into the femoral vein or other blood vessels for rapidly cooling the blood. Recent experiments have demonstrated that targeted organs may be cooled by delivering ice slurry through a small tube guided by endoscope to prevent ischemia during surgery.

Methods for producing phase-change ice particulate slurries are described in U.S. Pat. Nos. 6,244,052 and 6,413,444, both to Kasza. U.S. Pat. No. 6,244,052 relates to the production of phase-change ice particulate perfluorocarbon slurries and U.S. Pat. No. 6,413,444 relates to the production of phase-change particulate saline slurries. Again the teaching of both of these references in their entirety is incorporated by reference into the present disclosure.

A wide variety of delivery tubes, syringes and other delivery devices are commonly used to deliver fluids into the body. However, commercially available delivery devices are designed only for the delivery of single-phase fluids. These devices do not function satisfactorily to deliver phase-change particulate slurries such as those described in the above referenced patents. Currently available delivery devices often become plugged when used to deliver phase-change particulate slurries. Plugging occurs even though the cross sectional area of the slurry particles are significantly smaller than the cross sectional area of the flow path through the various delivery tubes, valves, fittings, insertion tip and any other components of the fluid delivery system.

A factor that contributes to plugging of the delivery device when delivering phase-change particulate slurries is the quality of the phase-change particulate slurry itself. Conventional phase-change slurries have dendritic ice particles which are highly elongated with very sharp appendages. Such particles are easily entangled and can begin to clump together. As clumps draw more and more particles they can begin to clog the components that form the flow path of the delivery device. With such particles clumping can occur at particulate loading levels as low as 5%.

The characteristics of the delivery device can also contribute to particulate clumping and eventual plugging of the delivery system flow path. Plugging can occur due to particle build up along the walls of delivery tubing or injector tips. Particles, especially dendritic particles, can become lodged against imperfections in the sidewall of the tubing and other components of the delivery systems. For example, particles can become trapped in minute cavities in the walls of the delivery tubing or against small protrusions extending from the walls into the flow path. Trapped particles rapidly lead to particulate build-up which can eventually occlude the slurry flow path.

Particle trapping is particularly prevalent at the interfaces between various flow path components. Component interfaces such as between a delivery tube and a control value, or between a delivery tube and the insertion tip, or simply between two tubes of different diameter, are often accompanied by sudden changes in the cross sectional area of the slurry flow path. For example, when two tubes of different diameter are joined, a significant reduction in the cross sectional area of the flow path occurs at the transition from the larger tube to the smaller tube. Slurry particles can become trapped against the forward facing step created by the smaller diameter tube. Again trapped particles can quickly grow into piles which eventually occlude the flow path. Particle build up leading to plugging is most serious at the injector tip of the delivery device. The injector represents the smallest cross section of the entire flow path.

Additional problems with conventional fluid delivery systems include, overly aggressive narrowing of the flow path, such as in a nozzle or insertion tip device, multi-stage tapering of the flow path, or sudden sharp changes in the direction of flow. All of these conditions can lead to trapped particles and subsequent particulate buildup and eventual plugging of the slurry flow path.

Some of the problems regarding plugging can be alleviated by improving the qualities of the phase-change ice particulate slurries. The U.S. Pat. Nos. 6,413,444 and 6,244,052 mentioned above address these problems by providing ice slurries having high quality smooth globular shaped particles that exhibit much lower plugging tendencies than conventional slurries. Such slurries allow for higher particle loading levels than previously possible. Nonetheless, even with these improved phase-change particulate slurries, plugging can still be a problem. Slurries having high ice particle load levels are highly desired to achieve maximal cooling from the smallest amount of coolant. In order to effectively deliver such improved heavily loaded slurries to targeted areas or organs within a patient's body,. new delivery mechanisms must be provided. The improved delivery mechanisms must far exceed the performance capabilities of presently available single-phase fluid delivery mechanisms, remaining free of obstructions at the highest particulate loading concentrations.

### BRIEF SUMMARY

The present invention relates to phase-change particulate ice slurry delivery systems for delivering ice slurry coolants to targeted areas of the body. A phase-change particulate ice slurry delivery system according to the invention includes a slurry reservoir and a conduit for conveying the slurry from the reservoir to the targeted area or organ of a patient. The delivery conduit may include multiple components. For example the delivery conduit may include an elongated section of flexible medical tubing, an insertion tip, and a transition fitting for adapting the medical tubing to the insertion tip. The slurry reservoir includes an exit port which allows for the out flow of slurry from the reservoir. The exit port forms an outwardly facing nipple that is insertable into a central lumen defined by the outer wall of the delivery tube. Slurry flows from the reservoir into the central lumen of the delivery tube. The internal interface between the exit port and the delivery tube is such that the cross sectional area of the flow path transitions from smaller to larger across the interface in the direction of flow away from the reservoir.

As noted above, the delivery conduit may comprise multiple components, including medical tubing, an insertion tip and one or more transition fittings. The interfaces between each component share the characteristic that the cross sectional area of the slurry flow path always transitions from smaller to larger across the interface in the direction of flow. With this geometry there are no forward facing steps at the interfaces which can trap particles and lead to plugging.

In some circumstances, such as in the insertion tip, and in some transition fittings, the flow path is necessarily narrowed. Whenever it is necessary to reduce the cross sectional area of the slurry flow path, the narrowing is accomplished via a gradual tapering of the flow path. Preferably the total included angle of taper does not exceed 20° relative to the central axis of the flow path.

Specially designed insertion tips are also provided. On example is a two port insertion tip. A first exit port is aligned axially with the central lumen extending through the insertion tip. The second exit port is off-axis in that the second exit port is formed in the side of the insertion tip. Preferably the second off-axis exit port is located within two lumen diameters of the first axially aligned exit port. A bevel surrounds the second exit port, forming an oblique surface to the direction of slurry flow at the downstream side of the second exit port. Accordingly slurry is caused to change direction gradually as it exits the second off axis exit port. Since there is no perpendicular impact surface, particulate will not become lodged against the downstream sidewall of the insertion tip that defines the second exit port.

A specially designed catheter for delivering slurry directly into blood vessels may also be employed as an insertion tip according to the invention. The specially designed catheter includes an input housing adapted to receive the output end of a tapered transition fitting. The transition fitting fits into a tapered bore formed in the input housing. Since the transition fitting is inserted into the housing and slurry flows from the transition fitting into the housing, the flow path maintains the proper geometry across interface between the transition fitting and the housing, transitioning from a smaller to a larger cross sectional area in the direction of flow.

The slurry reservoir may take on any number of different forms. For example the slurry reservoir may be a collapsible squeeze bag which is loaded with slurry and suspended from a rack. A plastic squeeze bottle has also been demonstrated, having an exit port formed in a threaded cap at the top of the bottle. Alternatively, the slurry reservoir may be a rigid container supplied with an agitator for maintaining uniform loading of the slurry particulate throughout the container.

The devices disclosed herein have proven effective for efficiently delivering phase-change particulate ice slurries to patients. The design of the various components, especially those that form the slurry flow path, is such that obstacles and protrusions into the flow path and other rapid changes in the flow path cross section are eliminated. Slurry particles flow freely from the reservoir to the targeted area without becoming trapped along the way at component interfaces and the like. Particles have no opportunity to accumulate and clog the system.

Other systems, methods, features and advantages of the invention will be, or will become, apparent to one with skill in the art upon examination of the following figures and detailed description. It is intended that all such additional systems, methods, features and advantages be included within this description, be within the scope of the invention, and be protected by the appended claims.

### BRIEF DESCRIPTION OF THE DRAWINGS

These and other features and advantages of the present invention will be better understood by reference to the following detailed description when considered in conjunction with the accompanying drawings, wherein:

FIG. 1 is a schematic illustration of a phase-change particulate ice slurry delivery system;

FIG. 2 is a cross section of a slurry reservoir exit port and an attached slurry delivery tube;

FIG. 3 is a detailed illustration of the distal end of a slurry delivery conduit including a delivery tube a transition fitting, and an insertion tip;

FIG. 4 is a cross section of the components shown in the detailed drawing of FIG. 3;

FIG. 5 is a detailed illustration of a two port insertion tip;

FIG. 6 is a detailed illustration of a clamp device for providing on/off flow control in a phase-change particulate ice slurry delivery system;

FIG. 7 is a cross section of a prior art single-phase solution delivering catheter;

FIG. 8 is a cross section of a phase-change particulate ice slurry delivering catheter, a transition fitting and a delivery tube;

FIG. 9 is an alternative embodiment of a phase-change particulate ice slurry delivery device wherein the slurry reservoir is a squeezable bottle;

FIG. 10 is a detailed cross section of a threaded cap for the squeeze bottle of FIG. 9;

FIG. 11 is another alternative embodiment of a phase-change particulate ice slurry delivery device wherein the slurry reservoir is a rigid container and includes an agitator for mixing slurry and a peristaltic pump for pumping slurry through a delivery tube;

FIG. 12 is yet another embodiment of a phase-change particulate ice slurry delivery system wherein the slurry reservoir as a syringe used in conjunction with a 14 gauge hypodermic needle.

### DETAILED DESCRIPTION OF THE DRAWINGS AND THE PRESENTLY PREFERRED EMBODIMENTS

The present invention relates to devices for delivering phase-change particulate ice slurries to specific targeted areas or internal organs of a patient's body. The fundamental components of a phase-change particulate ice slurry delivery device are a slurry reservoir for storing and transporting the phase-change particulate ice slurry; a conduit for delivering the slurry from the reservoir to the patient, and an injector device for injecting the slurry in the targeted area or organ of the patient. The various embodiments of the invention described herein are adapted to effectively deliver phase-change particulate ice slurries to patients without plugging of the slurry flow path resulting from unwanted accumulation of particles within the various flow path components and at the interfaces therebetween.

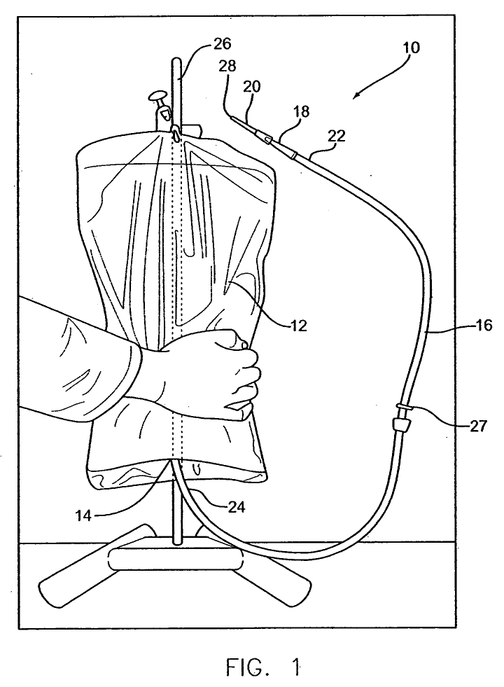

An embodiment of a phase-change particulate ice slurry delivery device 10 according to the invention is shown in FIG. 1. The device 10 includes a slurry reservoir 12, an elongate slurry delivery tube 16, and an insertion tip 20 for delivering slurry to the targeted area or organ. A transition fitting 18 is provided for attaching the insertion tip 20 to the distal end 22 of the slurry delivery tube 16. A proximal end 24 of the delivery tube 16 attaches to an exit opening 14 formed in a lower portion of the slurry reservoir 12.

According to the embodiment depicted in FIG. 1, the slurry reservoir 12 is provided by a collapsible squeeze bag filled with slurry. The flexible squeeze bag reservoir 12 may be suspended from a support rack 26. Slurry contained in the slurry reservoir 12 may be delivered to the target area or organ by positioning the insertion tip 20 near or directly within the targeted area or organ and manually squeezing the collapsible squeeze bag slurry reservoir 12. Squeezing the slurry reservoir 12 forces slurry out of the exit opening 14, through the slurry delivery tube 16, through the transition fitting 18, and out an exit port 28 formed at the end of the insertion tip 20.

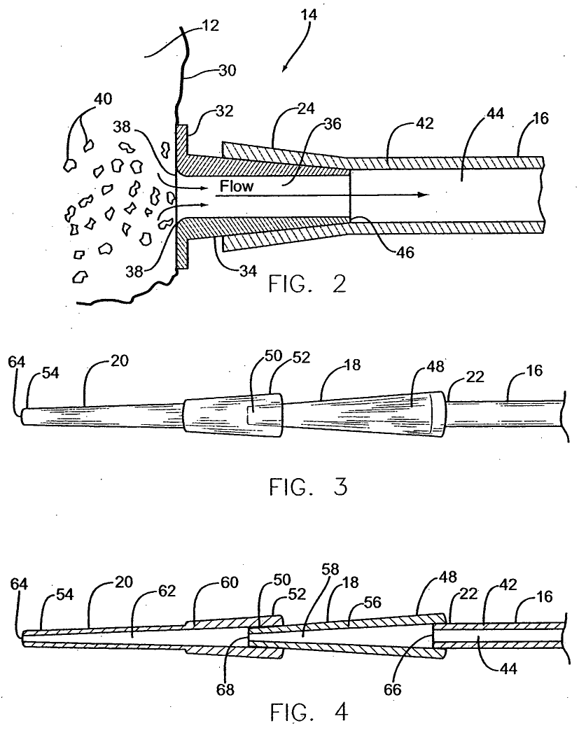

A detailed cross section of the interface between the slurry reservoir exit port 14 and the delivery tube 16 is shown in FIG. 2. The exit port 14 is formed in a side wall 30 of the collapsible squeeze bag slurry reservoir 12. A relatively rigid grommet-like base 32 surrounds the exit opening and is bonded to the side wall 30. A tapered extension 34 protrudes outwardly from the base 32 forming a nipple 34 at the lower end of the squeeze bag slurry reservoir 12. A hollow passage 36 extends through the nipple 34, forming an exit path for the slurry within the squeeze bag slurry reservoir 12. The entrance 38 to the hollow passage 36 is rounded with smooth surfaces. The rounded entrance 38 eliminates a flat surface which could otherwise trap particles and lead to plugging of the reservoir exit port 14. Accordingly, the slurry particles 40 flow smoothly into the hollow passage 36 and out of the squeeze bag slurry reservoir 12.

The proximal end of the delivery tube 16 fluidly connects to the nipple 34. The slurry delivery tube 16 is formed of an outer wall 42 that surrounds and defines a central lumen 44. The inner surface of the delivery tube wall 42 is substantially smooth, with no cavities or protrusions which can trap ice particles as the slurry flows through the delivery tube. The proximal end 24 of the slurry delivery tube 16 slides over the nipple 34, so that the hollow passage 36 through the nipple 34 communicates with the central lumen 44 defined by the delivery tube 16. The slurry delivery tube 16 frictionally engages the outer surface of the nipple 34 forming a snug-fit connection with the exit port 14. Slurry passes out of the reservoir 12, through the hollow passage 36, and into the central lumen 44 of the slurry delivery tube 16 and is delivered to the patient.

It must be noted that the tapered end of the nipple 34 is inserted into the central lumen 34 of the slurry delivery tube 16. The slurry delivery tube 16 is not inserted into the hollow passage 36. The diameter of the hollow passage 36 at the end of the nipple 34 is smaller than the diameter of the central lumen 44 of the slurry delivery tube 16. Therefore, the change in the cross sectional area of the flow path at the internal interface between the nipple 34 and the slurry delivery tube 16 transitions from smaller to larger in the direction of slurry flow away from the reservoir.

The internal interface between the exit port 14 and the deliver tube 16 is of critical importance to the effective delivery of phase-change particulate ice slurries. As slurry flows out of the squeeze bag slurry reservoir 12 through the hollow passage 36 and into the central lumen 44 of delivery tube 16, the slurry encounters no obstacles that could lead to particulate build up and eventual plugging of the flow path. The hollow passage 36 through the tapered extension 34 has a constant diameter (only the outer wall of the nipple 34 is tapered) as does the central lumen 44 of the delivery tube 16. Thus, the only change in the cross sectional area of the flow path occurs at the interior interface 46 where the flow path transitions the smaller diameter passage 36 through the nipple 34 to the relatively larger diameter central lumen 44 of the delivery tube 16. The step-like structure formed at the interface 46 faces away from the direction of flow. Thus the interface does not present a surface that can trap slurry particles or otherwise impede the flow of particles through the interface 46.

A feature of the present invention is that the interfaces between all components connected in the slurry flow path share this characteristic. There are no forward facing steps in the slurry flow path. Sudden changes in the cross sectional area of the slurry flow path are avoided as much as possible. Where they must occur they always transition from a smaller to a larger cross sectional area in the direction of flow. Where transitions from a larger to a smaller flow path must occur, such as within insertion tips or transitional fittings, the transitions occur gradually and smoothly. Preferably where a narrowing of the slurry flow path is required the total included angle of the tapered flow path will be less than about 20°. This will insure that particles do not bunch together in the area of taper and eventually clog the flow path.

Further illustrations of the proper interface between flow path components are found at the junctions between the distal end 22 of the slurry delivery tube 16 and the transition fitting 18 and between the transition fitting 18 and the insertion tip 20. A detailed view of these components is shown in FIG. 3. A cross section is shown in FIG. 4. The two views will be described together.

The distal end 22 of the delivery tube 16 is inserted into the flared tube receiving end 48 of the transition fitting 18. This connection forms the internal interface 66 between the delivery tube 16 and the transition fitting 18. Similarly, the exit end 50 of the transition fitting 18 is inserted into a receiving end 52 of the insertion tip 20. This connection forms the internal interface 68 between the transition fitting 18 and the insertion tip 20. The delivery tube 16 may be bonded to the transition fitting 18, or some other joining mechanism such as a snug-fit frictional connection may be provided to secure the transition fitting 18 to the distal end 22 of the delivery tube 16. Similar joining provisions may be applied between the transition fitting 18 and the insertion tip 20.

Like the delivery tube 16, the transition fitting 18 is formed of a generally cylindrical outer wall 56 which surrounds and defines a central lumen 58. When the transition fitting 18 is joined to the distal end 22 of the deliver tube 16, the central lumen of the delivery tube 16 is in fluid communication with the central lumen 58 of the transition fitting 18, thereby effectively extending the slurry flow path through the length of the transition fitting 18. At the internal interface 66 between the transition fitting 18 and the distal end 22 of the delivery tube, the diameter of the central lumen 58 of the transition fitting is greater than the diameter of the central lumen 44 of the delivery tube 16. Thus, the cross sectional area of the slurry flow path transitions from smaller to larger across the internal interface 66. The step-like structure formed at the interface 66 faces away from the direction of flow and does not present an obstacle to the flow of particles through the interface 66.

Unlike the delivery tube 16, the central lumen 58 of the transition fitting is tapered. The cross sectional area of the slurry flow is gradually reduced over the length of the transition fitting 18. In fact, the entire outer wall 56 of the transition fitting is tapered such that the outside diameter of the exit end 50 of the transition fitting 18 is substantially smaller than the outer diameter of the delivery tube 16. Thus, the exit end 50 of the transition fitting 18 may be inserted into the relatively small receiving end 52 of the insertion tip 20, whereas the distal end of the delivery tube 16 could not be.

Again, the insertion tip 20 may be bonded to the transition fitting, or a snug-fit frictional connection may be sufficient, or some of the connection mechanism may be employed to secure the insertion tip 20 to the transitional fitting 18. The insertion tip is formed by a tapered cylindrical outer wall 60 which surrounds and defines a central lumen 62. When the transition fitting 18 is inserted into the receiving end 50 of the insertion tip 20, the central lumen 58 of the transition fitting 18 is in fluid communication with the central lumen 62 of the insertion tip 20, thereby effectively extending the slurry flow path through the length of the insertion tip 20. At the internal interface 68 between the insertion tip 18 and the exit end 50 of the transition fitting 18, the diameter of the central lumen 62 of the insertion tip 20 is greater than the diameter of the central lumen 58 of the transition fitting 18. Thus, at the internal interface 68, the cross sectional area of the slurry flow path transitions from smaller to larger in the direction of flow. The step like structure formed at the interface 68 faces away from the direction of flow and does not present an obstacle to the flow of particles through the internal interface 68.

Like the transition fitting 18, the central lumen 62 of the insertion tip 20 is tapered. The slurry flow path is further narrowed along the length of the insertion tip 20. Preferably the amount of total included taper angle in the slurry flow path is less than about 20°. The entire insertion tip narrows to a relatively small point that can be inserted into various otherwise difficult to reach places that might not be accessible to wider instruments. Slurry exits the insertion tip 20 through a narrow nozzle-like exit port 64. The structure of the insertion tip provides for the directed flow of slurry from the slurry delivery apparatus 10.

Insertion tips having a single axial aligned aperture are preferred. Such insertion tips are the least likely to experience particulate build up and eventual plugging. However, in some applications dual port insertion tips are required. Dual ported insertion tips have the advantage that if one port is pushed against tissue, the tissue can block the delivery of slurry from that port. With two ports, even when one port is blocked the second port will continue to deliver slurry. Dual ports can also be advantageous during suctioning of slurry melt fluid for the same reasons.

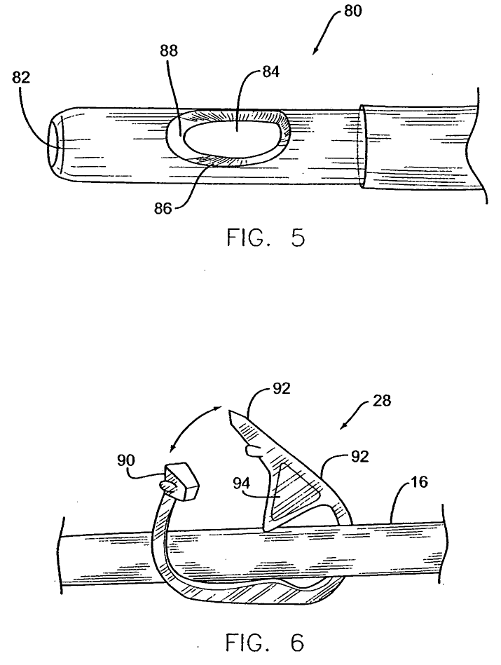

FIG. 5 shows a dual port insertion tip 80 in accord with the present invention. The dual port injector tip 80 includes a first axial aligned exit port 82 at the output end of the injector tip 80 and a second off-axis exit port 84 on the side of the injector tip 80. Preferably the second off-axis exit port 84 is near the first axial exit port 82. Empirical data have shown that a dual ported injector tip 80 performs best, with the least propensity toward clogging, when the second off-axis port 84 is located within 2-3 lumen diameters of the first axial port 82. Another significant characteristic of the second off-axis exit port 84 is the sharp bevel 86 formed around the perimeter of the exit port 84. The bevel 86 is especially critical on the downstream edge 88 of the second off-axis exit port 84. The sharp angle formed between the bevel and the slurry flow path gently alters the direction of the flow of a portion of the slurry flowing through the insertion tip 80. The sharp bevel does not present a surface that blocks the slurry particulate, but rather causes a gentle change of direction of the slurry flurry flowing out of the second off-axis exit port. Since the bevel 86 does not provide a significant obstacle to the continuous flow of slurry, ice particles will not accumulate and plug the flow path.

In the phase-change particulate ice slurry delivery of the present invention, on/off flow control depicted in FIG. 6 is provided by a pinch clamp valve 28. The pinch clamp valve 28 is a single-piece resilient spring-like clamp 96 configured to encircle the slurry delivery tube 16. A first clasp member 90 is formed at a first end of the single piece clamp 96, and a second clasp member 92 is formed at the opposite end. The pinch clamp valve 28 is closed to stop the flow of slurry through the slurry delivery tube 16 by manually pinching the single piece clamp 96 so that the second clasp member 92 engages the first clasp member 90. The first clasp member 90 and the second clasp member are configured such that the first clasp member 90 retains the second clasp member 92 unless and until the single piece clamp is manually opened. When the pinch clamp valve 28 is closed, actuator blade 94 engages the delivery tube 16, deforming the tube 16 such that the central lumen is pinched off, preventing the flow of slurry through the tube 16. When the clasp members 90, 92 are released, the single piece clamp 96 springs open. The actuator blade 94 is withdrawn from the delivery tube 16, opening the central lumen, allowing the resumption of slurry flow through the tube 16.

In some situations it is desirable to introduce phase-change particulate ice slurry into a blood vessel through a catheter. However, traditional catheters designed to deliver single phase solutions are ineffective for delivering phase-change particulate ice slurries. FIG. 7 shows a cross section of a traditional catheter 100. Catheter 100 includes a dual port inlet housing 102. A first inlet port 104 is configured to receive a medical tube for delivering fluid to the catheter, and which is to be injected into a patient. A sealing ring 108 and a check valve 110 are provided near the first inlet 104. The sealing ring acts to form a seal with the medical tubing delivering the fluid. The check valve 110 prevents the reverse flow of fluid from the patient back into the fluid delivery systems. The second inlet port 106 is provided so that additional fluid solutions (e.g. additional medications) may be merged with the primary solution which is delivered to the patient via the first inlet port 104. The catheter tip 112 is a long narrow flexible tube that may be inserted a significant distance into the patient's body. For example catheter tip 112 may be adapted for insertion into a patient's femoral vein. The tip can be up to 30 cm. in length.

The inlet housing 102 of a traditional catheter such as catheter 100 is especially prone to plugging when used to deliver phase-change particulate ice slurries. The sealing ring 108 and especially the check valve 110 present obstacles to the slurry flow path which can trap particles and lead to plugging. Surfaces 116 and 118 associated with the second inlet port 106 and the entrance to the catheter tip 112 itself can also trap particles and lead to plugging. A new inlet housing was necessary to adapt traditional catheters for delivering phase-change particulate ice slurries.

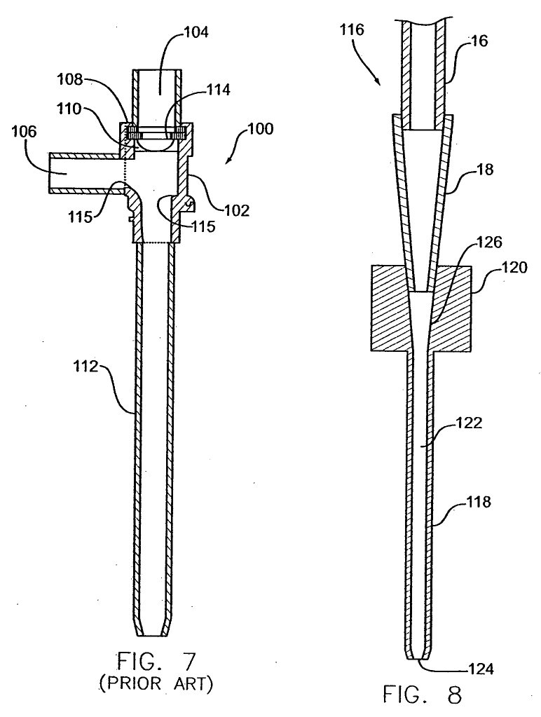

FIG. 8 shows a catheter 116 designed in accordance with principles of the present invention. Catheter 116 includes a catheter tip 118 and an inlet housing 120. The catheter tip 118 is substantially the same as catheter tip 112 shown in FIG. 7. Catheter tip 118 comprises a long flexible tube defining a central lumen 122, and having an axially aligned exit port 124 at the distal end. The inlet housing 120 comprises an enlarged collar integrally formed with the catheter tip 118. A tapered entrance bore 126 extends through the inlet housing 120 and communicates with and is axially aligned with the central lumen 122. The tapered bore 126 is configured to receive the distal end of a transition fitting 18 which in turn is attached to the distal end of a slurry delivery tube 16 as has already been described. The slurry delivery tube 16 and the transition fitting may be substantially the same as those described with reference FIGS. 3 and 4. The only alterations that might be necessary for using the same delivery tube 16 and transition fitting 18 is that it may be desirable to change the sizes of the delivery tube and the transition fitting in order to alter the volume of slurry delivered to the catheter 116. Further, it may be desirable to change the size and shape of the transition piece 18 to provide a more effective fit between the transition fitting 18 and the catheter inlet housing 120. It is important to note, that at the interface between the transition fitting 18 to the inlet housing 120 there are no restrictions in the slurry flow path. The change in cross sectional area of the flow path across the interface is from smaller to larger in the direction of slurry flow. There are no surfaces or obstacles that will tend to trap particles, and lead to particle accumulation and plugging.

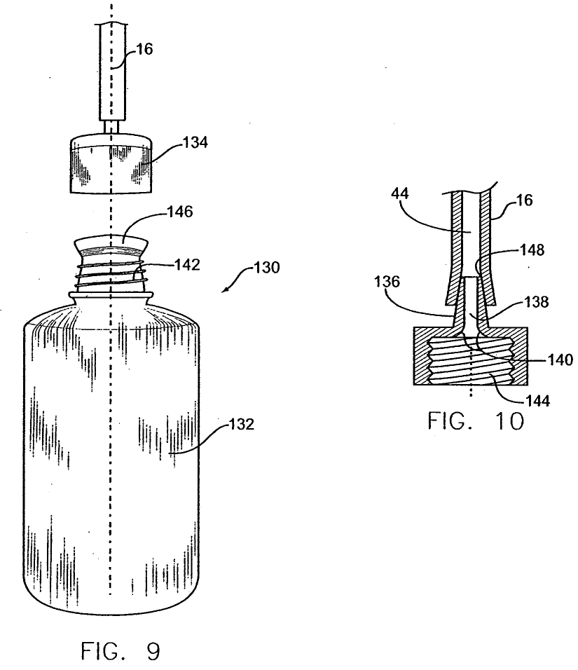

An alternative embodiment of a slurry reservoir 130 is shown in FIGS. 9 and 10. According to this embodiment the slurry reservoir comprises a more structured container such as a squeezable plastic bottle 132. An advantage of a squeeze bottle reservoir 132 is that it is more portable and the bottle may be frequently shaken in order to maintain an even distribution of slurry particles throughout the slurry. The bottle includes a relatively large threaded opening 146 for receiving slurry, and a threaded cap 134 with a smaller tapered exit port 136 for dispensing slurry. With the threaded cap 134 removed, squeeze bottle 132 may be filled with phase-change particulate ice slurry. Once the squeeze bottle 132 is filled, the threaded cap 134 is rotated onto the threaded opening 142. The internal threads 144 of the cap 134 engage the external threads 142 of the bottle to substantially seal the threaded opening, but for the tapered exit port 136. Slurry may then be pumped out of the squeeze bottle 132 through the tapered exit port 136 by manually squeezing the bottle 132.

The tapered exit port 136 has all the same characteristics of the exit port 14 of the flexible squeeze bag 12 described above with reference to FIGS. 1 and 2. The tapered exit port 136 has an external cone-shaped taper and a substantially constant diameter exit passage 138. The entrance 140 to the central passage 138 is rounded to prevent particles from accumulating as slurry is pushed out of the squeeze bottle 132. The external taper of the exit port 136 is adapted to be inserted into the central lumen 44 a slurry delivery tube 16 in the same manner as described above with regard to the exit port 14 of the squeeze bag. The cross sectional area of the slurry flow path increases across the interface between the exit port 136 to the slurry delivery tube 16 in the direction of flow. Accordingly, the step like structure 148 formed at the internal interface 148 points away from the direction of flow and does not present a surface that will block slurry particles and lead to plugging.

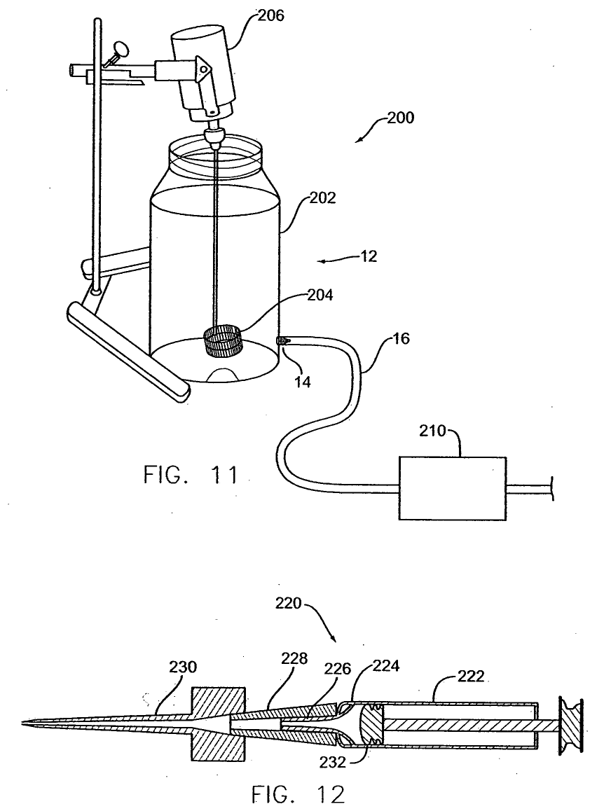

FIG. 11 shows another embodiment of a phase-change particulate ice slurry delivery system 200. In broad aspect, the delivery system 200 of FIG. 11 is the same as the system 10 in FIG. 1, comprising a slurry reservoir 12, a slurry delivery tube 16 and an insertion tip (not shown in FIG. 11). The slurry reservoir of system 200, however, is a rigid container 202. Reservoir 202 for example may be a four liter cylindrical tank with a contoured bottom as shown. An agitator 204 is provided for mixing and stirring the phase-change particulate ice slurry stored in the rigid container 202. An actuator 206 is provided for driving the agitator. Absent continuous mixing, a stagnant phase-change particulate ice slurry begins to stratify. The lighter less dense ice particles tend to float to the top of the container 202, while the heavier solution tends to settle to the bottom. Such non-uniform loading of the slurry can lead to delivery problems, and can adversely affect the cooling properties of the slurry. The actuator may be a commercially available laboratory mixer such as Stirpak Model #5002-20, running at a speed in the range between 9-900 RPM. The agitator may be a 1-gallon paint mixing blade such as Hyde Tools #43440. The mixing motor and mixing blade assembly may be mounted above the rigid bottle container by a ring stand such as Stirpak Model 50001-92. The mixing paddle should be located off center to minimize vortexing in the container and effectively stir the entire contents.

The rigid container 202 includes an exit port 14 substantially identical to that described above with regard to the squeeze bag reservoir 12 and the squeeze bottle reservoir 132. The slurry delivery tube 16 fits over the exit port 14 also as previously described. In the previous embodiments, however, slurry was pumped through the delivery tube 16 by manually squeezing a flexible reservoir to force the slurry through the delivery tube 16. This manual pumping mechanism is not available with the rigid reservoir 202 of system 200. Accordingly, an in-line tube pump 210 is provided to pump the phase-change particulate ice slurry through the delivery tube 16. In-line tube pump 210 may be a 3 roller peristaltic pump such as Masterflex L/S Easy-Load pump head 7720-62 with Economy Digital Drive #07524-40. Preferably the in line tube pump will be capable of pumping 0-50% loaded ice slurries at a rate of between 10-700 ml/min.

Yet another embodiment of a phase-change particulate ice slurry delivery system 220 is shown in FIG. 12. In this embodiment the slurry reservoir and pumping mechanism are provided by a syringe 222. A manually operated plunger 224 forces slurry out of the tapered outlet port 226 integrally formed with the body of the syringe 222. The tapered outlet port entrance central lumen has a smooth contoured inlet 232. The tapered outlet port may be inserted into a slurry delivery tube such as that employed with previously described embodiments, or the exit port may be inserted into a transition fitting 228 as shown in FIG. 11, or the tapered output port 226 may be inserted directly into an insertion tip, a catheter, or some other end device for directing the flow of slurry to a targeted area. In the arrangement shown in FIG. 12 the tapered exit port 226 is inserted into a transition fitting 228 which is in turn inserted into the receiving end of a fourteen gauge hypodermic needle 230. An advantage of system 220 is that the syringe 222, in conjunction with the hypodermic needle 230, can be used to inject slurry into very targeted areas of the patient's body, such as directly into a kidney or other organ. The syringe can also be used to remove melted slurry. As with the other embodiments, all flow path components are configured such that the interfaces between components transition from a smaller cross sectional area larger cross sectional area in the direction of flow across the various interfaces. Accordingly there are no forward facing projections into the flow path that can block the flow of particles and lead to plugging.

It should be understood, and therefore included within the scope of this invention that the phase particulate delivery tubes and insertion tip devices can be replaced with a wide variety of different embodiments or devices including, automated or manual features. For example instead of using the manually operated slurry delivery containers the delivery tube can be interfaced with a slurry pumping system, for example a roller tubing pump. While the principles of the present invention have been made clear in illustrative embodiments, it will be immediately obvious to those skilled in the art many modifications of structure, arrangement, proportions, the elements, materials and components used in the practice of the invention and otherwise, which are particularly adapted to specific environments without departing from those principles. The following claims are intended to embrace and cover any and all such modifications with the limits only of the true spirit scope of the invention.

What is claimed is:

1. A phase-change particulate slurry delivery system comprising:
a slurry reservoir having an exit port; and
a multi-component conduit defining a central lumen fluidly connected to the exit port;
the exit port and the central lumen of the multi-component conduit defining a flow path for delivering slurry from the reservoir to a desired location;
the exit port and the multi-component conduit defining a first flow path interface, and one or more junctions forced between individual components of the multi-component conduit forming at least one subsequent flow path interface;
the interfaces configured such that the flow path transitions from a smaller cross sectional are to a larger cross sectional area across the interfaces in the direction of flow away from the reservoir.

2. The system of claim 1 wherein the flow path is gently tapered through at least one of the components of the multi-component conduit, the flow path narrowing in the direction of flow.

3. The system of claim 2 wherein the flow path taper is less than above 20°.

4. The system of claim 1 wherein the multi-component conduit comprises a flexible delivery tube and an insertion tip connected at a distal end of the delivery tube.

5. The system of claim 4 wherein the insertion tip defines a tapered central lumen extending through the insertion tip, the central lumen having a receiving end and a discharge port, both axially aligned with the central lumen, the receiving end having an inner diameter greater than an inner diameter of the discharge port.

6. The system of claim 5 wherein the delivery tube has an outer diameter less than the inner diameter of the receiving end of the central lumen of the insertion tip so that the delivery tube may be inserted directly into the central lumen of the insertion tip.

7. The system of claim 6 wherein the delivery tube has an outer diameter greater than the inner diameter of the receiving end of the central lumen of the insertion tip, the multi-component conduit further comprising a transition fitting for adapting the delivery tube for use with the insertion tip.

8. The system of claim 7 wherein the transition fitting has a tapered body and defines a central lumen, the tapered body of the transition fitting having an output end with an outer diameter smaller than the inner diameter of the receiving end of the central lumen of the insertion tip so that the output end of the transition fitting may be inserted into the receiving end of the central lumen of the insertion tip, and the central lumen of the transition fitting having a diameter greater than the outer diameter of the delivery tube so that the delivery tube may be inserted into the central lumen of the transition fitting.

9. The system of claim 5 wherein the insertion tip defines a central lumen, the insertion tip having a first exit port axially aligned with the central lumen, and a second off-axis exit port formed in a sidewall of the insertion tip, the second off-axis exit port forming a sharp bevel at an edge of the off-axis exit port facing into the outwardly directed slurry flow.

10. The system of claim 5 wherein the insertion device comprises a catheter having a tapered receiving port housing for receiving a correspondingly tapered transition fitting connected to the distal end of the delivery tube.

11. The system of claim 1 wherein the slurry reservoir exit port comprises a tapered nipple extending outwardly from the reservoir and adapted to be inserted into a central lumen of the multi-component conduit.

12. The system of claim 11 wherein the reservoir comprises a collapsible squeeze bag having a phase-change particulate slurry stored therein.

13. The system of claim 11 wherein the reservoir comprises a squeezable bottle, and where the tapered nipple is formed on a threaded cap enclosing the squeeze bottle.

14. The system of claim 11 wherein the reservoir is a substantially rigid container and further comprises an agitator for mixing the slurry to maintain uniform particulate loading throughout the slurry.

15. The system of claim 14 further comprising a peristaltic pump for pumping slurry through the multi-component conduit.

16. The system of claim 11 comprises a syringe having a manually operated plunger for forcing slurry out through the exit port which is positioned at the distal end of the syringe.

17. The system of claim 16 wherein the multi-component conduit comprises a hypodermic needle and a transition fitting for adapting the syringe to hypodermic needle.

18. A phase-change ice particulate slurry delivery tubing and insertion device, comprising:
a phase-change particulate container having an exit aperture; and
an slurry coolant delivery system, including,
an elongated delivery tube in fluid communication at one of its ends with the exit aperture, the elongated delivery tube having an inner lumen to convey the ice particle slurry from the container in a flow direction,
a transition fitting connected to the elongated delivery tube; and
an insertion tip that introduces the ice particle slurry into the body through an exit aperture in the insertion tip, the insertion tip having an inner lumen in fluid communication with the inner lumens of the transition fitting and delivery tube(s) which are insertable in a human being,
wherein the inner lumen of the transition fitting and the delivery tube(s) in the slurry coolant delivery system has a gradual taper from a larger diameter to a smaller diameter in the flow direction and wherein the inner lumens of the elongated delivery tube, transition fitting, and the insertion tip are free of obstructions of the ice particle slurry in the flow direction, the delivery system attached to the exit aperture of the phase-change particulate container.

19. The ice particulate slurry coolant delivery device of claim 18 wherein the surfaces of the inner lumens are smooth.

20. The ice particulate slurry coolant delivery device of claim 18 wherein inner lumen of the transition fitting has a cross sectional diameter having a gradual uniform taper from a larger to smaller diameter.

21. The ice particulate slurry coolant delivery device of claim 18 wherein the exit aperture is configured in the insertion tip such that the ice slurry exits the insertion tip at an oblique angle relative to the inner lumen of the insertion tip.

22. The ice particulate slurry coolant delivery device of claim 18 further comprising a clamp valve disposed to control the flow of the ice particle slurry through the elongated delivery tube.

23. An ice slurry coolant delivery device, comprising:
an ice slurry coolant receptacle, the ice slurry coolant receptacle having an aperture;
a delivery tube having a first diameter placed over the aperture; and
a transition fitting having a receiving end having a diameter larger than the first diameter and an output end having second diameter smaller than the first diameter, the transition fitting connects to the delivery tube with the delivery tube being insertable into the transition fitting receiving end forming a downstream facing step at the connection, whereby the ice slurry coolant material does not plug the opening of the transition fitting.

24. An ice particulate slurry coolant delivery device, comprising:
an ice slurry particulate container having an exit opening;
a delivery tube in fluid communication at one of its ends with the exit aperture of the container, the first delivery tube having and inner lumen to convey the ice particle slurry from the container in a flow direction; and
an insertion tip that introduces the ice particle slurry into the body through at least one exit aperture in the insertion tip, with one aperture formed on a side wall of the insertion tip, the side wall of the insertion tip at the downstream edge of the aperture being beveled to provide a sharp edge to an oncoming flow of ice particle slurry.

25. The ice particulate slurry coolant delivery device of claim 24 wherein the bevel on the downstream edge of the aperture comprises a shaved portion of the wall of the insertion tip.

26. The ice particulate slurry coolant delivery device of claim 24 wherein the surface of the inner lumen is smooth.

27. The ice particulate slurry coolant delivery device of claim 24 wherein the inner lumen of the insertion tip has a cross sectional diameter having a gradual uniform taper from a larger to smaller diameter.

28. The ice particulate slurry coolant delivery device of claim 24 further comprising a clamp valve disposed to control the flow of the ice particle slurry through the elongated delivery tube.

29. An ice particulate slurry coolant delivery device, comprising:
an ice slurry particulate container having an exit opening;
a first delivery tube in fluid communication at one of its ends with the exit aperture of the container, the first delivery tube having an inner lumen to convey the ice particle slurry from the container in a flow direction;
a second delivery tube, the second delivery tube having an inner lumen to convey the ice particle slurry the flow direction, the inner lumen of the second delivery tube being smaller than the inner lumen of the first delivery tube;
a transition fitting having an inner lumen and connected between the first delivery tube and the second delivery tube, the cross sectional diameter of the inner lumen of the transition fitting having a gradual uniform taper from a larger to smaller diameter, the first delivery tube inserted into a first end of the transition fitting such that a first downstream facing step is formed in the flow direction, a second end of the transition fitting inserted into the second delivery tube such that a second downstream facing step is formed in the flow.

30. The ice particulate slurry coolant delivery device of claim 29 wherein the second delivery tube comprises an insertion tip that introduces the ice particle slurry into the body wherein the inner lumen of the insertion tip has a cross sectional diameter having a gradual uniform taper from a larger to smaller diameter.

31. The ice particulate slurry coolant delivery device of claim 30 wherein the surfaces of the inner lumen of the first delivery tube, the transition fitting, and the insertion tip are smooth.

32. The ice particulate slurry coolant delivery device of claim 31 wherein the exit aperture is configured in the insertion tip such that the ice slurry exits the insertion tip at an oblique angle relative to the inner lumen of the insertion tip.

33. The ice particulate slurry coolant delivery device of claim 29 further comprising a clamp valve disposed to control the flow of the ice particle slurry through the elongated delivery tube.
